(19) United States
(12) Patent Application Publication
(10) Pub. No.: US 2005/0229332 A1
(43) Pub. Date: Oct. 20, 2005

(54) Charging cosmetic makeup composition for keratin fibers

(75) Inventors: Sophie Bodelin, Vanves (FR); Delphine Lepeltier, Montrouge (FR)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(73) Assignee: L'OREAL

(21) Appl. No.: 11/048,911

(22) Filed: Feb. 3, 2005

(30) Foreign Application Priority Data
Feb. 3, 2004 (FR) 04 50198

(51) Int. Cl. A61K 7/13

(52) U.S. Cl. 8/405

(57) ABSTRACT

The present disclosure relates to a keratin fiber coating composition comprising, in a cosmetically acceptable medium, at least one ionic surfactant and at least one nonionic surfactant with an HLB of greater than or equal to 8 at 25° C., wherein the composition has: a solids content of greater than or equal to 40% by weight, and
an adhesive profile of less than or equal to 3, wherein the adhesive profile is the ratio of the adhesive power at the time when 20% of the composition has evaporated (AP_{20%}), to the adhesive power at T₀ (AP_{T0}, T₀ corresponding to the time of application of the composition). The present disclosure also relates to the use of such a composition to obtain a charging makeup result on keratin fibers.

The present disclosure relates to a cosmetic composition for coating keratin fibers.

The composition as disclosed herein may be a makeup composition, such as a mascara, a makeup base for keratin fibers, or a base coat, a composition to be applied over makeup, also known as a top coat, or a composition for treating keratin fibers.

In one embodiment, the composition as disclosed herein is a leave-in composition. For example, the composition as disclosed herein can be a mascara.

As used herein, the term “mascara” means a composition intended to be applied to the eyelashes: it may be an eyelash makeup composition, an eyelash makeup base, a composition to be applied over a mascara, also known as a top coat, or a cosmetic eyelash treatment composition. The mascara is, for example, intended for human eyelashes, but also for false eyelashes.

Eye makeup compositions, such as “mascaras” for the eyelashes, generally comprise a wax or a mixture of waxes dispersed using at least one surfactant in an aqueous phase also comprising polymers and pigments.

It is generally through the qualitative and quantitative choice of the waxes and polymers that the desired application specificities for makeup compositions are adjusted, for instance their fluidity, their covering power and/or their curling power. Thus, it is possible to produce various compositions, which, when applied, for example, to the eyelashes, induce a variety of effects such as lengthening, curling and/or thickening (also called a “charging effect”).

The present disclosure is directed to a composition that is useful for producing a heavy makeup result on keratin fibers such as the eyelashes, which is also known as “charging makeup.” As used herein, the term “keratin fibers” includes the hair, the eyelashes, the eyebrows, and also synthetic wigs and false eyelashes.

With the makeup compositions that are currently available, a charging effect is generally obtained by superimposing several coats of a makeup composition onto keratin fibers such as the eyelashes. Moreover, in the case of the eyelashes, this charging effect is often associated with an aggregation of several eyelashes together, i.e., a non-individualization of the eyelashes.

For obvious reasons, it would be advantageous to obtain this thickening (charging) effect in a single application while at the same time obtaining good separation of the eyelashes.

To do this, it would be advantageous to have, for example, a makeup composition that is sufficiently concentrated in dry matter to significantly charge the eyelashes from the very first time they come into contact with the composition, and that also allows each eyelash to be coated with the solids content. However, a problem of lack of fluidity may arise. The makeup composition may become too thick to apply and may no longer have the deformability required for its uniform application over the entire surface of the eyelashes.

It has thus been found, unexpectedly, that it is possible to prepare compositions with a dry extract (i.e., solids content) of greater than or equal to 40% by weight using, in these compositions, a specific emulsifying system combining at least one ionic surfactant and at least one nonionic surfactant with an HLB (hydrophilic-lipophilic balance) of greater than or equal to 8 at 25° C., wherein the compositions have, for example, an adhesive power that allows good attachment to keratin fibers at the time of application, without making the deposit too tacky while drying, in order to provide easy application.

Disclosed herein is thus a keratin fiber coating composition comprising, in a cosmetically acceptable medium, at least one ionic surfactant and at least one nonionic surfactant with an HLB of greater than or equal to 8 at 25° C. wherein the composition has:
a solids content of greater than or equal to 40% by weight, and
an adhesive profile of less than or equal to 3, wherein the adhesive profile is the ratio of the adhesive power at the time when 20% of the composition has evaporated (AP_{20%}), to the adhesive power at T₀ (AP_{T0}), wherein T₀ means the time of application of the composition.

The present disclosure is also directed to a process for making up keratin fibers, comprising applying the composition disclosed herein to the keratin fibers.

The present disclosure also relates to the use of the composition as disclosed herein to obtain a charging makeup result on keratin fibers such as on the eyelashes and the eyebrows.

Disclosed herein is also the use of the combination of at least one ionic surfactant and at least one nonionic surfactant with an HLB of greater than or equal to 8 at 25° C. to obtain a keratin fiber coating composition with a solids content of greater than or equal to 40% by weight and an adhesive profile of less than or equal to 3, wherein the composition allows a charging makeup result on keratin fibers such as the eyelashes and the eyebrows.

As used herein, the term “charging” makeup means thick and volumizing makeup of keratin fibers such as the eyelashes.

The keratin fiber coating composition as disclosed herein is, for example, not solid. As used herein, the term “solid” composition means a composition that does not flow whatever the stress applied to the composition at room temperature. This type of solid composition is, for example, a solid mascara, also called cake mascara. To apply such solid mascara on the eyelashes, it is necessary to decake it with a mascara brush impregnated with water, and then the mixture of mascara and water is applied on the eyelashes with the brush.

The Solids Content

The solids content, i.e., the content of non-volatile matter, may be measured in various ways; examples that may be mentioned include oven-drying methods, drying methods by exposure to infrared radiation and also chemical methods by titration of the water according to Karl Fischer.

The solids content, commonly referred to as the “dry extract,” of the compositions disclosed herein, is measured, for example, by heating the sample with infrared rays with a wavelength of 2 μm to 3.5 μm. The substances contained in the compositions that have a high vapour pressure evaporate under the effect of this radiation. Measurement of the weight loss of the sample makes it possible to determine the “dry extract” of the composition. These measurements are performed using an LP16 commercial infrared desiccator from Mettler. This technique is fully described in the machine documentation supplied by Mettler.

The measuring protocol is as follows:

About 1 g of the composition is spread onto a metal crucible. After introducing this crucible into the desiccator, it is subjected to a set temperature of 120° C. for one hour. The wet mass of the sample, corresponding to the initial mass, and the dry mass of the sample, corresponding to the mass after exposure to the radiation, are measured using a precision balance.

The solids content is calculated in the following manner:
Dry extract=100×(dry mass/wet mass).

The compositions as disclosed herein have a solids content of greater than or equal to 40% by weight, such as greater than or equal to 41% by weight, further such as greater than or equal to 42% by weight and even further such as greater than or equal to 45% by weight, relative to the total weight of the composition. In one embodiment, the compositions as disclosed herein have a solids content of up to 55% by weight, such as up to 50% by weight, relative to the total weight of the composition.

Adhesive Profile of the Composition

The adhesive profile of the composition is measured at room temperature, in accordance with the following protocol, using the texture analyzer under the name TA-TX2i from the company Rheo, on a sample of the composition spread onto a glass plate in the form of a film 300 μm thick.

The test comprises placing a cylindrical elastomer probe of 6 mm in diameter in contact with the product.

The probe is applied (speed=0.1 mm/s) with a force of 1N onto the product (for 10 s) and is then withdrawn (speed=1 mm/s). The force required to withdraw the probe is measured. The tack (adhesive power) corresponds to the integral of the curve of the force as a function of time for the portion of the curve of the force with negative value (withdrawal force).

The adhesive power is expressed in N.s.

The measurement is taken at T₀ (AP_{T0}) and at 20% evaporation (AP_{20%}). The adhesive profile is equal to AP_{20%}/AP_{T0}. The AP_{T0} is the measurement of the adhesive power at T₀, directly after the composition has been spread onto the pre-tared glass plate and the deposited mass measured using an AX204 precision balance from the company Mettler-Toledo. The AP_{20%} is the measurement of the adhesive power after 20% of the composition applied to the glass plate has evaporated off, i.e., when the film spread onto the glass plate has lost 20% of its initial mass: the mass of the film, determined by calculation, then corresponds to 80% of its initial mass. The AP_{20%} is measured once the balance displays this value.

The composition as disclosed herein has an adhesive profile ranging, for example, from 0.1 to 3, such as from 0.5 to 2.7, further such as from 1 to 2.5, and even further such as from 1.2 to 2.3. In one embodiment, the composition as disclosed herein has an adhesive profile ranging from 1.5 to 2.

Emulsifying System

As disclosed herein, an emulsifier appropriately chosen to obtain an oil-in-water or wax-in-water emulsion is used. For example, an emulsifier having an HLB (hydrophilic-lipophilic balance) at 25° C. according to Griffin, of greater than or equal to 8 is used.

The HLB value according to Griffin is defined in J. Soc. Cosm. Chem. 1954 (volume 5), pages 249-256.

The compositions as disclosed herein may comprise at least one emulsifying surfactant present in an amount ranging, for example, from 0.1% to 40% by weight, such as from 0.3% to 20% by weight, relative to the total weight of the composition.

These emulsifying surfactants may be chosen from nonionic, anionic, cationic and amphoteric surfactants. Reference may be made to the “Encyclopedia of Chemical Technology, Kirk-Othmer”, volume 22, pp. 333-432, 3rd edition, 1979, Wiley, for the definition of the properties and (emulsifying) functions of surfactants, for example, at pages 347-377, for anionic, amphoteric and nonionic surfactants.

As disclosed herein, the emulsifying system comprises at least one ionic surfactant and at least one nonionic surfactant with an HLB of greater than or equal to 8 at 25° C.

Nonionic Surfactant with an HLB of Greater Than or Equal to 8

As non-limiting examples of nonionic surfactants with an HLB of greater than or equal to 8 at 25° C. that may be used, alone or as a mixture, in the makeup compositions disclosed herein, mention may be made of:
oxyethylenated and/or oxypropylenated ethers (which may comprise from 1 to 150 oxyethylene and/or oxypropylene groups) of glycerol;
oxyethylenated and/or oxypropylenated ethers (which may comprise from 1 to 150 oxyethylene and/or oxypropylene groups) of fatty alcohols (such as those of C₈-C₂₄ alcohol, for example, those of C₁₂-C₁₈ alcohol), such as oxyethylenated cetearyl alcohol ether comprising 30 oxyethylene groups (CTFA name “Ceteareth-30”) and the oxyethylenated ether of the mixture of C₁₂-C₁₅ fatty alcohols comprising 7 oxyethylene groups (CTFA name “C₁₂₋₁₅ Pareth-7” sold under the name “Neodol 25-7®” by Shell Chemicals);
fatty acid esters (such as those of a C₈-C₂₄ acid, for example, those of a C₁₆-C₂₂ acid) of polyethylene glycol (which may comprise from 1 to 150 ethylene glycol units), such as PEG-50 stearate and PEG-40 monostearate sold under the name Myrj 52P by the company ICI Uniquema;
fatty acid esters (such as those of a C₈-C₂₄ acid, for example, those of a C₁₆-C₂₂ acid) of oxyethylenated and/or oxypropylenated glyceryl ethers (which may comprise from 1 to 150 oxyethylene and/or oxypropylene groups), for instance PEG-200 glyceryl monostearate sold under the name “Simulsol 220 TM” by the company SEPPIC; glyceryl stearate polyethoxylated with 30 ethylene oxide groups, for instance the product Tagat S sold by the company Goldschmidt, glyceryl oleate polyethoxylated with 30 ethylene oxide groups, for instance the product Tagat O sold by the company Goldschmidt, glyceryl cocoate polyethoxylated with 30 ethylene oxide groups, for instance the product Varionic LI 13 sold by the company Sherex, glyceryl isostearate polyethoxylated with 30 ethylene oxide groups, for instance the product Tagat L sold by the company Goldschmidt, and glyceryl laurate polyethoxylated with 30 ethylene oxide groups, for instance the product Tagat I from the company Goldschmidt;
fatty acid esters (such as those of a C₈-C₂₄ acid, for example, those of a C₁₆-C₂₂ acid) of oxyethylenated and/or oxypropylenated sorbitol ethers (which may comprise from 1 to 150 oxyethylene and/or oxypropylene groups), for instance polysorbate 60 sold under the name “Tween 60” by the company Uniqema;
dimethicone copolyol, such as the product sold under the name “Q2-5220” by the company Dow Corning;
dimethicone copolyol benzoate (Finsolv SLB 101 and 201 by the company Finetex);
copolymers of propylene oxide and of ethylene oxide, also known as EO/PO polycondensates;
and mixtures thereof.

The EO/PO polycondensates are, for example, chosen from copolymers comprising polyethylene glycol and polypropylene glycol blocks, for instance polyethylene glycol/polypropylene glycol/polyethylene glycol triblock polycondensates. These triblock polycondensates have, for example, the following chemical structure:
H—(O—CH₂—CH₂)ₐ—(O—CH(CH₃)—CH₂)_{b}—(O—CH₂—CH₂)ₐ—OH,
wherein a ranges from 2 to 120 and b ranges from 1 to 100.

The EO/PO polycondensate has a weight-average molecular weight ranging, for example, from 1000 to 15 000 such as from 2000 to 13 000. In one embodiment, the EO/PO polycondensate has a cloud point, at 10 g/l in distilled water, of greater than or equal to 20° C. such as greater than or equal to 60° C. The cloud point is measured according to ISO standard 1065. As the EO/PO polycondensates that may be used herein, mention may be made, for example, of the polyethylene glycol/polypropylene glycol/polyethylene glycol triblock polycondensates sold under the name “Synperonic”, for instance “Synperonic PE/L44” and “Synperonic PE/F127”, by the company ICI, and mixtures thereof.

One or more nonionic surfactants with an HLB of less than 8 at 25° C. may, where appropriate, be combined with the at least one nonionic surfactant with an HLB of greater than or equal to 8.

As non-limiting examples of the non-ionic surfactants with an HLB of less than 8 at 25° C., mention may be made of:
saccharide esters and ethers, such as sucrose stearate, sucrose cocoate and sorbitan stearate, and mixtures thereof, for instance Arlatone 2121 sold by the company ICI;
fatty acid esters (such as those of a C₈-C₂₄ acid, for example, those of a C₁₆-C₂₂ acid) of polyol, for example, fatty acid esters of glycerol or of sorbitol, such as glyceryl stearate, glyceryl stearate such as the product sold under the name Tegin M by the company Goldschmidt, glyceryl laurate such as the product sold under the name Imwitor 312 by the company Hüls, polyglyceryl-2 stearate, sorbitan tristearate or glyceryl ricinoleate; and
the mixture of cyclomethicone/dimethicone copolyol sold under the name “Q2-3225C” by the company Dow Corning.

The amount of the at least one nonionic surfactant with an HLB of greater than or equal to 8 is generally adjusted so as to obtain a composition having the parameters as defined above. The determination of this amount falls within the competence of a person skilled in the art.

For example, the amount of the at least one nonionic surfactant with an HLB of greater than or equal to 8 may range from 0.01% to 40% by weight, such as from 0.1% to 25% by weight, further such as from 0.2% to 15% by weight, and even further such as from 0.4% to 10% by weight, relative to the total weight of the composition.

Ionic Surfactant

The ionic surfactants used herein may be anionic or cationic. In one embodiment, at least one anionic surfactant is used.

Among the anionic surfactants that may be used herein, mention may be made, for example, of:
C₁₆-C₃₀ fatty acid salts, such as those derived from amines, for instance triethanolamine stearate and 2-amino-2-methyl-1,3-propanediol stearate;
polyoxyethylenated fatty acid salts, such as those derived from amines or alkali metal salts, and mixtures thereof;
phosphoric esters and salts thereof, such as “DEA oleth-10 phosphate” (Crodafos N 10N from the company Croda);
sulfosuccinates such as “Disodium PEG-5 citrate lauryl sulfosuccinate” and “Disodium ricinoleamido MEA sulfosuccinate”;
alkyl ether sulfates, such as sodium lauryl ether sulfate;
isethionates;
acylglutamates such as “Disodium hydrogenated tallow glutamate” (Amisoft HS-21 R sold by the company Ajinomoto), and
mixtures thereof.

In one embodiment, at least one of triethanolamine stearate and 2-amino-2-methyl-1,3-propanediol stearate is used. Triethanolamine stearate and 2-amino-2-methyl-1,3-propanediol stearate are generally obtained by simple mixing of stearic acid with triethanolamine and with 2-amino-2-methyl-1,3-propanediol, respectively.

Examples of the cationic surfactants that may be mentioned include:
alkylimidazolidiniums, such as isostearylethylimidonium ethosulfate, and
ammonium salts, such as N,N,N-trimethyl-1-docosanaminium chloride (behentrimonium chloride).

The compositions as disclosed herein may also comprise one or more amphoteric surfactants, for instance N-acylamino acids such as N-alkylaminoacetates and disodium cocoamphodiacetate, and amine oxides such as stearamine oxide, or alternatively silicone surfactants, for instance dimethicone copolyol phosphates such as the product sold under the name “Pecosil PS 100” by the company Phoenix Chemical.

In general, the compositions as disclosed herein may comprise, for example, from 0.01% to 30% by weight such as from 0.1% to 15% by weight, and further such as from 0.5% to 10% by weight, of the ionic surfactants, relative to the total weight of the composition.

Wax

The composition as disclosed herein may comprise a wax or a mixture of waxes.

The wax used herein is generally a lipophilic compound that is solid at room temperature (25° C.), with a solid/liquid reversible change of state, having a melting point of greater than or equal to 30° C., which may be up to 120° C.

By bringing the wax to the liquid form (melting), it is possible to make it miscible with oils and to form a microscopically uniform mixture, but on cooling the mixture to room temperature, recrystallization of the wax in the oils of the mixture is obtained.

For example, the waxes that may be used herein may have a melting point of greater than about 45° C., such as greater than or equal to 50° C. and further such as greater than or equal to 55° C.

The melting point of the wax may be measured using a differential scanning calorimeter (DSC), for example, the calorimeter sold under the name DSC 30 by the company Mettler.

The measuring protocol is as follows:

A sample of 15 mg of product placed in a crucible is subjected to a first temperature rise ranging from 0° C. to 120° C., at a heating rate of 10° C./minute, it is then cooled from 120° C. to 0° C. at a cooling rate of 10° C./minute and is finally subjected to a second temperature increase ranging from 0° C. to 120° C. at a heating rate of 5° C./minute. During the second temperature increase, the variation of the difference in power absorbed by the empty crucible and by the crucible containing the sample of product is measured as a function of the temperature. The melting point of the compound is the temperature value corresponding to the top of the peak of the curve representing the variation in the difference in absorbed power as a function of the temperature.

The waxes that may be used in the compositions disclosed herein are chosen from waxes that are solid and rigid at room temperature, of animal, plant, mineral or synthetic origin, and mixtures thereof.

The wax may also have a hardness ranging, for example, from 0.05 MPa to 30 MPa such as from 6 MPa to 15 MPa. The hardness is determined by measuring the compressive strength, measured at 20° C. using the texturometer sold under the name TA-TX2i by the company Rheo, equipped with a stainless-steel cylinder 2 mm in diameter travelling at a measuring speed of 0.1 mm/s, and penetrating into the wax to a penetration depth of 0.3 mm.

The measuring protocol is as follows:

The wax is melted at a temperature equal to the melting point of the wax +20° C. The molten wax is cast in a container 30 mm in diameter and 20 mm deep. The wax is recrystallized at room temperature (25° C.) over 24 hours and is then stored for at least 1 hour at 20° C. before performing the hardness measurement. The value of the hardness is the maximum compressive strength measured divided by the area of the texturometer cylinder in contact with the wax.

Hydrocarbon-based waxes such as beeswax, lanolin wax and Chinese insect waxes; rice wax, carnauba wax, candelilla wax, ouricury wax, esparto grass wax, cork fibre wax, sugar cane wax, Japan wax and sumach wax; montan wax, microcrystalline waxes, paraffins and ozokerite; polyethylene waxes, the waxes obtained by Fisher-Tropsch synthesis and waxy copolymers, and also esters thereof, may, for example, be used.

The waxes obtained by catalytic hydrogenation of animal or plant oils comprising linear or branched C₈-C₃₂ fatty chains, may, for example, also be used.

Among these oils, mention may be made, for example, of hydrogenated jojoba oil, isomerized jojoba oil such as the trans-isomerized partially hydrogenated jojoba oil manufactured or sold by the company Desert Whale under the trade name Iso-Jojoba-50®, hydrogenated sunflower oil, hydrogenated castor oil, hydrogenated coconut oil and hydrogenated lanolin oil, bis(1,1,1-trimethylolpropane) tetrastearate sold under the name “Hest 2T-4S” by the company Heterene, and bis(1,1,1-trimethylolpropane) tetrabehenate sold under the name Hest 2T-4B by the company Heterene.

Silicone waxes and fluoro waxes may, for example, also be used.

Further for example, the wax obtained by hydrogenation of olive oil esterified with stearyl alcohol, sold under the name “Phytowax Olive 18 L 57”, or the waxes obtained by hydrogenation of castor oil esterified with cetyl alcohol, sold under the name “Phytowax Ricin 16L64 and 22L73” by the company Sophim, may also be used. Such waxes are described in French patent application FR-A-2 792 190.

In one embodiment, the compositions as disclosed herein may comprise at least one “tacky” wax, i.e., a wax with a tack of greater than or equal to 0.7 N.s and a hardness of less than or equal to 3.5 MPa.

Using a tacky wax may, for example, make it possible to obtain a cosmetic composition that applies easily to keratin fibers, attaches well to the keratin fibers and leads to the formation of a smooth, uniform and thickening makeup result.

The tacky wax used may, for example, have a tack ranging from 0.7 N.s to 30 N.s, such as greater than or equal to 1 N.s, for example, from 1 N.s to 20 N.s, further such as greater than or equal to 2 N.s, for example, from 2 N.s to 10 N.s and further, for example, from 2 N.s to 5 N.s.

The tack of the wax is determined by measuring the change in force (compression force or stretching force) as a function of time, at 20° C., using the texturometer sold under the name “TA-TX2i®” by the company Rheo, equipped with a conical acrylic polymer spindle forming an angle of 45°.

The measuring protocol is as follows:

The wax is melted at a temperature equal to the melting point of the wax+10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the tack.

The texturometer spindle is displaced at a speed of 0.5 mm/s then penetrates the wax to a penetration depth of 2 mm. When the spindle has penetrated the wax to a depth of 2 mm, the spindle is held still for 1 second (corresponding to the relaxation time) and is then withdrawn at a speed of 0.5 mm/s.

During the relaxation time, the force (compression force) decreases greatly until it becomes zero, and then, during the withdrawal of the spindle, the force (stretching force) becomes negative and then rises again to the value 0. The tack corresponds to the integral of the curve of the force as a function of time for the part of the curve corresponding to negative values of the force (stretching force). The tack value is expressed in N.s.

The tacky wax that may be used generally has, for example, a hardness of less than or equal to 3.5 MPa, such as from 0.01 MPa to 3.5 MPa, further such as from 0.05 MPa to 3 MPa, and even further such as from 0.1 MPa to 2.5 MPa.

The hardness is measured according to the protocol described above.

Tacky waxes that may be used include a C₂₀-C₄₀ alkyl (hydroxystearyloxy)stearate (wherein the alkyl group comprises from 20 to 40 carbon atoms), alone or as a mixture, such as a C₂₀-C₄₀ alkyl 12-(12′-hydroxystearyloxy)stearate, of formula (II): 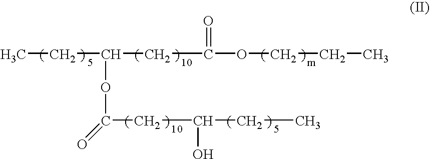
wherein m is an integer ranging from 18 to 38, or a mixture of compounds of formula (II).

Such a wax is, for example, sold under the names “Kester Wax K 82 P®” and “Kester Wax K 80 P®” by the company Koster Keenan.

The waxes mentioned above generally have, for example, a starting melting point of less than 45° C.

The composition as disclosed herein may comprise a total wax content ranging, for example, from 1% to 50% by weight, such as from 5% to 40% by weight, further such as from 10% to 35% by weight, and even further such as from 10% to 30% by weight, relative to the total weight of the composition.

As disclosed herein, it is also possible to use waxes supplied in the form of small particles having a size, expressed as the mean “effective” volume diameter D[4,3], ranging, for example, from 0.5 to 30 micrometers, such as from 1 to 20 micrometers, further such as from 5 to 10 micrometers in size, which are used herein as “microwaxes”.

The particle sizes may be measured by various techniques; mention may be made, for example, of light-scattering techniques (dynamic or static), Coulter counter methods, sedimentation rate measurements (related to the size via Stoke's lax) and microscopy. These techniques make it possible to measure a particle diameter and, for some of them, a particle size distribution.

The sizes and size distributions of the particles in the compositions as disclosed herein are, for example, measured by static light scattering using a commercial granulometer such as the MasterSizer 2000 from Malvern. The data are processed on the basis of the Mie scattering theory. This theory, which is exact for isotropic particles, makes it possible to determine an “effective” particle diameter in the case of non-spherical particles. This theory is described, for example, in the publication by Van de Hulst, H. C., “Light Scattering by Small Particles”, Chapters 9 and 10, Wiley, New York, 1957.

The microwax used herein is characterized by its mean “effective” diameter by volume D[4,3], defined in the following manner:

$$D [ 4 , 3 ] = \frac{{\sum }_{i} {V}_{i} \cdot {d}_{i}}{{\sum }_{i} {V}_{i}}$$

wherein Vᵢ is the volume of the particles with an effective diameter dᵢ. This parameter D[4,3] is described, for example, in the technical documentation of the granulometer.

The measurements are performed at 25° C. on a dilute particle dispersion, obtained from the microwax in the following manner: 1) dilution by a factor of 100 with water, 2) homogenization of the solution, 3) standing of the solution for 18 hours, 4) recovery of the whitish uniform supernatant.

The “effective” diameter is obtained by taking a refractive index of 1.33 for water and a mean refractive index of 1.42 for the particles.

Among the microwaxes that may be used in the compositions as disclosed herein, mention may be made, for example, of carnauba microwaxes, such as the product sold under the name “MicroCare 350®” by the company Micro Powders, synthetic microwaxes, such as the product sold under the name “MicroEase 114S®” by the company Micro Powders, microwaxes comprising a mixture of carnauba wax and polyethylene wax, such as the products sold under the name “Micro Care 300®” and “310®” by the company Micro Powders, microwaxes comprising a mixture of carnauba wax and synthetic wax, such as the product sold under the name “Micro Powders 325®” by the company Micro Powders, polyethylene microwaxes, such as the products sold under the names “Micropoly 200®”, “220®”, “220L®”, and “250S®” by the company Micro Powders, and polytetrafluoroethylene micropowders, such as the products sold under the names “Microslip 519®” and “519L®” by the company Micro Powders.

The waxes (including the tacky wax) may be present in the form of an aqueous microdispersion of wax. The term “aqueous microdispersion of wax” means an aqueous dispersion of wax particles in which the size of the wax particles is less than or equal to 1 μm.

Wax microdispersions are stable dispersions of colloidal wax particles, and are described, for example, in “Microemulsions Theory and Practice”, L. M. Prince Ed., Academic Press (1977) pages 21-32.

For example, these wax microdispersions may be obtained by melting the wax in the presence of a surfactant, and optionally in the presence of a portion of water, followed by gradual addition of hot water with stirring. The intermediate formation of an emulsion of the water-in-oil type is observed, followed by a phase inversion, with final production of a microemulsion of the oil-in-water type. On cooling, a stable microdispersion of solid wax colloidal particles is obtained.

The wax microdispersions may also be obtained by stirring the mixture of wax, surfactant and water using stirring tools such as ultrasound, high-pressure homogenizers or turbomixers.

The particles of the wax microdispersion have, for example, mean sizes of less than 1 μm (such as ranging from 0.02 μm to 0.99 μm) and, for example, less than or equal to 0.5 μm (such as ranging from 0.06 μm to 0.5 μm).

These particles consist essentially of a wax or a mixture of waxes. However, they may comprise a small proportion of oily and/or pasty fatty additives, a surfactant and/or a common liposoluble additive/active agent.

When the wax or the mixture of waxes is present in the compositions as disclosed herein in the form of an aqueous dispersion of particles, the size of the particles, i.e., the mean “effective” volume diameter D[4,3] as defined above, may be, for example, less than or equal to 1 μm such as less than or equal to 0.75 μm.

The wax particles may have varied shapes. For example, they may be spherical.

In one embodiment, the composition is free of isomerized jojoba oil such as the trans-isomerized partially hydrogenated jojoba oil manufactured or sold by the company Desert Whale under the trade name Iso-Jojoba-50®. Specifically, without being bound by this theory, it is believed that the isomerized jojoba oil, which recrystallizes at room temperature, is not favorable for obtaining a composition of uniform and smooth appearance.

Consequently, further disclosed herein is a keratin fiber coating composition comprising, in a cosmetically acceptable medium, at least one ionic surfactant and at least one nonionic surfactant with an HLB of greater than or equal to 8 at 25° C. wherein the composition has a solids content of greater than or equal to 40% by weight, is free of isomerized jojoba oil, and is not solid. The at least one ionic surfactant and the at least one nonionic surfactant with an HLB of greater than or equal to 8 at 25° C. are as described above.

In one embodiment, the composition disclosed herein comprises at least one paraffin wax, for example, in an amount of greater than or equal to 1% by weight, such as greater than or equal to 5% by weight, and further such as greater than or equal to 10% by weight, relative to the total weight of the composition.

Thus, further disclosed herein is a keratin fiber coating composition comprising, in a cosmetically acceptable medium, at least one ionic surfactant, at least one nonionic surfactant with an HLB of greater than or equal to 8 at 25° C. and at least one paraffin wax, wherein the composition has a solids content of greater than or equal to 40% by weight, and is not solid. The at least one ionic surfactant and the at least one nonionic surfactant with an HLB of greater than or equal to 8 at 25° C. are as described above.

The compositions as disclosed herein may further comprise at least one film-forming polymer.

As used herein, the term “film-forming polymer” means a polymer capable, by itself or in the presence of an auxiliary film-forming agent, of forming a continuous film that adheres to a support such as a keratin material, for instance the eyelashes.

The at least one film-forming polymer may be present in the composition as disclosed herein in a solids content ranging, for example, from 0.1% to 60% by weight, such as from 0.5% to 40% by weight, and further such as from 1% to 30% by weight, relative to the total weight of the composition.

Among the film-forming polymers that may be used in the composition as disclosed herein, mention may be made, for example, of synthetic polymers of radical-mediated type or polycondensate type, and polymers of natural origin, and mixtures thereof.

As used herein, the term “radical-mediated film-forming polymer” means a polymer obtained by polymerization of monomers comprising at least one unsaturation, such as ethylenic unsaturation, wherein each monomer is capable of homopolymerizing (unlike polycondensates).

The film-forming polymers of radical-mediated type may be chosen, for example, from vinyl polymers or copolymers, such as acrylic polymers.

The vinyl film-forming polymers can result from the polymerization of monomers comprising at least one ethylenic unsaturation and at least one acidic group and/or esters of these acidic monomers and/or amides of these acidic monomers.

Monomers comprising at least one acid group which may be used include, for example, α,β-ethylenic unsaturated carboxylic acids such as acrylic acid, methacrylic acid, crotonic acid, maleic acid or itaconic acid. (Meth)acrylic acid and crotonic acid are, for example, used. In one embodiment, (meth)acrylic acid is used.

The esters of acidic monomers are chosen, for example, from (meth)acrylic acid esters (also known as (meth)acrylates), such as (meth)acrylates of an alkyl, for example, a C₁-C₃₀ alkyl, such as a C₁-C₂₀ alkyl, (meth)acrylates of an aryl, such as a C₆-C₁₀ aryl, and (meth)acrylates of a hydroxyalkyl, such as a C₂-C₆ hydroxyalkyl.

Among the alkyl (meth)acrylates that may be mentioned, examples include methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl (meth)acrylates that may be mentioned, examples include hydroxyethyl acrylate, 2-hydroxypropyl acrylate, hydroxyethyl methacrylate and 2-hydroxypropyl methacrylate.

Among the aryl (meth)acrylates that may be mentioned, examples include benzyl acrylate and phenyl acrylate.

The (meth)acrylic acid esters that may be used are, for example, alkyl (meth)acrylates.

As disclosed herein, the alkyl group of the esters may be either fluorinated or perfluorinated, i.e., some or all of the hydrogen atoms of the alkyl group are substituted with fluorine atoms.

Examples of amides of the acid monomers that may be mentioned include (meth)acrylamides, such as N-alkyl(meth)acrylamides, for example, of a C₂-C₁₂ alkyl. Among the N-alkyl(meth)acrylamides that may be mentioned, examples include N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. For example, these monomers may be polymerized with acid monomers and/or esters thereof and/or amides thereof, such as those mentioned above.

Examples of vinyl esters that may be mentioned include vinyl acetate, vinyl neodecanoate, vinyl pivalate, vinyl benzoate and vinyl t-butylbenzoate. Styrene monomers that may be mentioned include styrene and α-methylstyrene.

Among the film-forming polycondensates that may be mentioned, examples include polyurethanes, polyesters, polyesteramides, polyamides, epoxyester resins and polyureas.

The polyurethanes may be chosen from anionic, cationic, nonionic or amphoteric polyurethanes, polyurethane-acrylics, polyurethane-polyvinylpyrrolidones, polyester-polyurethanes, polyether-polyurethanes, polyureas and polyurea-polyurethanes, and mixtures thereof.

The polyesters may be obtained, in a known manner, by polycondensation of dicarboxylic acids with polyols, such as diols.

The dicarboxylic acid may be aliphatic, alicyclic or aromatic. Examples of such acids that may be mentioned include: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalene-dicarboxylic acid and 2,6-naphthalenedicarboxylic acid. These dicarboxylic acid monomers may be used alone or as a combination of at least two dicarboxylic acid monomers. Among these monomers, phthalic acid, isophthalic acid and terephthalic acid may, for example, be used.

The diol may be chosen from aliphatic, alicyclic and aromatic diols. The diol used is, for example, chosen from ethylene glycol, diethylene glycol, triethylene glycol, 1,3-propanediol, cyclohexanedimethanol and 4-butanediol. Other polyols that may be used include glycerol, pentaerythritol, sorbitol and trimethylolpropane.

The polyesteramides may be obtained in a manner analogous to that of the polyesters, by polycondensation of diacids with diamines or amino alcohols. Diamines that may be used include, for example, ethylenediamine, hexamethylenediamine and meta- or para-phenylenediamine. An amino alcohol that may be used is, for example, monoethanolamine.

The polyester may also comprise at least one monomer bearing at least one group —SO₃M, wherein M is chosen from a hydrogen atom, an ammonium ion NH₄⁺ and a metal ion such as an Na⁺, Li⁺, K⁺, Mg²⁺, Ca²⁺, Cu²⁺, Fe²⁺ or Fe³⁺ ion. A difunctional aromatic monomer comprising such a group —SO₃M may, for example, be used.

The aromatic nucleus of the difunctional aromatic monomer also comprising a group —SO₃M as described above may be chosen, for example, from benzene, naphthalene, anthracene, biphenyl, oxybiphenyl, sulfonylbiphenyl and methylenebiphenyl nuclei. Among the difunctional aromatic monomers also comprising a group —SO₃M, mention may be made, for example, of sulfoisophthalic acid, sulfoterephthalic acid, sulfophthalic acid, 4-sulfonaphthalene-2,7-dicarboxylic acid.

The copolymers used are, for example, those based on isophthalate/sulfoisophthalate, such as copolymers obtained by condensation of diethylene glycol, cyclohexanedimethanol, isophthalic acid and sulfoisophthalic acid.

The composition may comprise at least one water-soluble film-forming polymer chosen, for example, from:
proteins, for instance proteins of plant origin such as wheat or soybean proteins; proteins of animal origin such as keratins, for example keratin hydrolysates and sulfonic keratins;
anionic, cationic, amphoteric or nonionic chitin or chitosan polymers;
cellulose polymers such as hydroxyethylcellulose, hydroxypropylcellulose, methylcellulose, ethylhydroxyethylcellulose and carboxymethylcellulose, and also quaternized cellulose derivatives;
vinyl polymers, for instance polyvinylpyrrolidones, copolymers of methyl vinyl ether and of maleic anhydride, the copolymer of vinyl acetate and of crotonic acid, copolymers of vinylpyrrolidone and of vinyl acetate; copolymers of vinylpyrrolidone and of caprolactam; polyvinyl alcohol;
polymers of natural origin, optionally modified, such as:
gum arabics, guar gum, xanthan derivatives and karaya gum;
alginates and carrageenans;
glycoaminoglycans, and hyaluronic acid and its derivatives;
shellac resin, sandarac gum, dammar resins, elemi gums and copal resins;
deoxyribonucleic acid;
mucopolysaccharides such as hyaluronic acid and chondroitin sulfate, and mixtures thereof.

In one embodiment, the film-forming polymer may be a polymer dissolved in a liquid organic medium of the composition comprising oils or organic solvents such as those described below (in which case, the film-forming polymer is a liposoluble polymer).

Examples of the liposoluble polymers that may be mentioned include copolymers of a vinyl ester (wherein the vinyl group is directly linked to the oxygen atom of the ester group and the vinyl ester comprises a radical chosen from saturated, linear or branched hydrocarbon-based radicals of 1 to 19 carbon atoms, linked to the carbonyl of the ester group) and of at least one other monomer, which may be a vinyl ester (different from the vinyl ester already present), an α-olefin (comprising from 8 to 28 carbon atoms), an alkyl vinyl ether (the alkyl group of which comprises from 2 to 18 carbon atoms) or an allylic or methallylic ester (comprising a radical chosen from saturated, linear or branched hydrocarbon-based radicals of 1 to 19 carbon atoms, linked to the carbonyl of the ester group).

These copolymers may be crosslinked using crosslinking agents that may be either of the vinylic type or of the allylic or methallylic type, such as tetraallyloxyethane, divinylbenzene, divinyl octanedioate, divinyl dodecanedioate and divinyl octadecanedioate.

Examples of these copolymers which may be mentioned include the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Examples of the liposoluble film-forming polymers which may also be mentioned include liposoluble copolymers, such as those resulting from the copolymerization of vinyl esters comprising from 9 to 22 carbon atoms or of alkyl acrylates or methacrylates, wherein the alkyl radicals comprise from 10 to 20 carbon atoms.

Such liposoluble copolymers may be chosen, for example, from polyvinyl stearate, polyvinyl stearate crosslinked with the aid of divinylbenzene, of diallyl ether or of diallyl phthalate copolymers, polystearyl (meth)acrylate, polyvinyl laurate and polylauryl (meth)acrylate copolymers, it being possible for these poly(meth)acrylates to be crosslinked with the aid of ethylene glycol dimethacrylate or tetraethylene glycol dimethacrylate.

The liposoluble copolymers defined above are known and are described, for example, in French patent application FR-A-2 232 303; they may have a weight-average molecular weight ranging, for example, from 2000 to 500 000 such as from 4000 to 200 000.

Among the liposoluble film-forming polymers which may be used herein, mention may also be made, for example, of polyalkylenes such as copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP) such as copolymers of vinylpyrrolidone and of C₂ to C₄₀ alkene such as C₃ to C₂₀ alkene. Among the VP copolymers which may be used herein, mention may be made, for example, of the copolymers of VP/vinyl acetate, VP/ethyl methacrylate, butylated polyvinylpyrrolidone (PVP), VP/ethyl methacrylate/ methacrylic acid, VP/eicosene, VP/hexadecene, VP/triacontene, VP/styrene or VP/acrylic acid/lauryl methacrylate.

The film-forming polymer may also be present in the composition in the form of particles dispersed in an aqueous phase or in a non-aqueous solvent phase (liquid organic medium of the composition), which is generally known as a latex or pseudolatex. The techniques for preparing these dispersions are well known to those skilled in the art.

Aqueous dispersions of film-forming polymers which may be used are the acrylic dispersions sold under the names “Neocryl XK-90®”, “Neocryl A-1070®”, “Neocryl A-1090®”, “Neocryl BT-62®”, “Neocryl A-1079®” and “Neocryl A-523®” by the company Avecia-Neoresins, “Dow Latex 432®” by the company Dow Chemical, “Daitosol 5000 AD®” or “Daitosol 5000 SJ” by the company Daito Kasey Kogyo; “Syntran 5760” by the company Interpolymer or the aqueous dispersions of polyurethane sold under the names “Neorez R-981®” and “Neorez R-974®” by the company Avecia-Neoresins, “Avalure UR-405®”, “Avalure UR-410®”, “Avalure UR-425®”, “Avalure UR-450®”, “Sancure 875®”, “Sancure 861®”, “Sancure 878®” and “Sancure 2060®” by the company Goodrich, “Impranil 85®” by the company Bayer and “Aquamere H-1511® by the company Hydromer; the sulfopolyesters sold under the brand name “Eastman AQ®” by the company Eastman Chemical Products, vinyl dispersions, for instance “Mexomer PAM” and also acrylic dispersions in isododecane, for instance “Mexomer PAP” by the company Chimex.

The composition disclosed herein may further comprise at least one plasticizer that promotes the formation of a film with the film-forming polymer. Such a plasticizer may be chosen from any compound known to those skilled in the art as being capable of satisfying the desired function.

Cosmetically Acceptable Medium

The cosmetically acceptable medium of the presently disclosed composition comprises, for example, an aqueous phase, which can form the continuous phase of the composition.

The aqueous phase may comprise water; it may also comprise a mixture of water and at least one water-miscible solvent (water miscibility of greater than 50% by weight at 25° C.), for instance lower monoalcohols comprising from 1 to 5 carbon atoms, such as ethanol and isopropanol, glycols comprising from 2 to 8 carbon atoms, such as propylene glycol, ethylene glycol, 1,3-butylene glycol and dipropylene glycol, C₃-C₄ ketones and C₂-C₄ aldehydes, and mixtures thereof.

The aqueous phase (water and, optionally, the water-miscible solvent) may be present in an amount ranging, for example, from 0.1% to 95% by weight such as from 1% to 80% by weight, relative to the total weight of the composition.

In one embodiment, the aqueous phase is present in an amount of greater than or equal to 20% by weight, such as greater than or equal to 30% by weight, further such as greater or equal to 40% by weight, and even further such as greater than or equal to 50% by weight, relative to the total weight of the composition.

The compositions disclosed herein may also comprise ingredients commonly used in the field of makeup for keratin fibers.

For example, the composition disclosed herein may comprise at least one oil.

The at least one oil may be chosen from volatile oils and non-volatile oils, and mixtures thereof. In one embodiment, the presently disclosed composition comprises at least one volatile oil.

As used herein, the term “volatile oil” means an oil that is capable of evaporating on contact with the skin or a keratin fiber in less than one hour, at room temperature and atmospheric pressure. The at least one volatile organic solvent and the at least one volatile oils disclosed herein are volatile organic solvents and cosmetic oils that are liquid at room temperature, with a non-zero vapour pressure at room temperature and atmospheric pressure, ranging, for example, from 0.13 Pa to 40 000 Pa (10⁻³ to 300 mmHg), such as from 1.3 Pa to 13 000 Pa (0.01 to 100 mmHg), and further such as from 1.3 Pa to 1300 Pa (0.01 to 10 mmHg). The term “non-volatile oil” means an oil that remains on the skin or the keratin fiber at room temperature and atmospheric pressure for at least several hours and that has, for example, a vapour pressure of less than 10⁻³ mmHg (0.13 Pa).

These oils may be hydrocarbon-based oils, silicone oils or fluoro oils, or mixtures thereof.

As used herein, the term “hydrocarbon-based oil” means an oil mainly comprising hydrogen and carbon atoms and optionally oxygen, nitrogen, sulfur and/or phosphorus atoms. The volatile hydrocarbon-based oils may be chosen, for example, from hydrocarbon-based oils comprising from 8 to 16 carbon atoms, such as branched C₈-C₁₆ alkanes, for instance C₈-C₁₆ isoalkanes of petroleum origin (also known as isoparaffins), such as isododecane (also known as 2,2,4,4,6-pentamethylheptane), isodecane and isohexadecane, for example, the oils sold under the trade names Isopar or Permetyl, branched C₈-C₁₆ esters and isohexyl neopentanoate, and mixtures thereof. Other volatile hydrocarbon-based oils, for instance petroleum distillates, such as those sold under the name Shell Solt by the company Shell, may also be used. The volatile solvent is chosen, for example, from volatile hydrocarbon-based oils comprising from 8 to 16 carbon atoms, and mixtures thereof.

The volatile oils that may also be used include, for example, volatile silicones, for instance volatile linear or cyclic silicone oils, such as those with a viscosity ≦8 centistokes (8×10⁻⁵ m²/s) and, for example, comprising from 2 to 7 silicon atoms, these silicones optionally comprising at least one group chosen from alkyl and alkoxy groups comprising from 1 to 10 carbon atoms. Among the volatile silicone oils that may be used herein, mention may be made, for example, of octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane and dodecamethyl pentasiloxane, and mixtures thereof.

Volatile fluorinated solvents such as nonafluoromethoxybutane or perfluoromethylcyclopentane may, for example, also be used.

The volatile oil may be present in the composition as disclosed herein in an amount ranging, for example, from 0.1% to 60% by weight, such as from 0.1% to 30% by weight, relative to the total weight of the composition.

The composition may also comprise at least one non-volatile oil chosen, for example, from non-volatile hydrocarbon-based oils, silicone oils, and fluoro oils.

Non-volatile hydrocarbon-based oils that may, for example, be mentioned include:
hydrocarbon-based oils of plant origin, such as triglycerides comprising fatty acid esters of glycerol, the fatty acids of which may have varied chain lengths from C₄ to C₂₄, these chains possibly being linear or branched, and saturated or unsaturated; these oils are chosen, for example, from wheatgerm oil, sunflower oil, grapeseed oil, sesame seed oil, maize oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, marrow oil, blackcurrant oil, evening primrose oil, millet oil, barley oil, quinoa oil, rye oil, safflower oil, candlenut oil, passion flower oil and musk rose oil; or alternatively caprylic/capric acid triglycerides such as those sold by Stearineries Dubois or those sold under the names Miglyol 810, 812 and 818 by Dynamit Nobel,
synthetic ethers comprising from 10 to 40 carbon atoms;
linear or branched hydrocarbons of mineral or synthetic origin, such as petroleum jelly, polydecenes, hydrogenated polyisobutene such as parleam, and squalane, and mixtures thereof;
synthetic esters such as oils of formula R₁COOR₂ wherein R₁ is chosen from linear and branched fatty acid residues comprising from 1 to 40 carbon atoms and R₂ is chosen from branched hydrocarbon-based chains comprising from 1 to 40 carbon atoms, provided that the number of carbon atoms in R₁+R₂≧10, such as, purcellin oil (cetostearyl octanoate), isopropyl myristate, isopropyl palmitate, C₁₂-C₁₅ alkyl benzoate, hexyl laurate, diisopropyl adipate, isononyl isononanoate, 2-ethylhexyl palmitate, isostearyl isostearate, alkyl or polyalkyl octanoates, decanoates or ricinoleates such as propylene glycol dioctanoate; hydroxylated esters such as isostearyl lactate and diisostearyl malate; and pentaerythritol esters;
fatty alcohols that are liquid at room temperature, comprising a branched and/or unsaturated carbon-based chain comprising from 12 to 26 carbon atoms, for instance octyldodecanol, isostearyl alcohol, oleyl alcohol, 2-hexyldecanol, 2-butyloctanol and 2-undecylpentadecanol;
higher fatty acids such as oleic acid, linoleic acid and linolenic acid;
and mixtures thereof.

The non-volatile silicone oils that may be used in the composition as disclosed herein may, for example, be non-volatile polydimethylsiloxanes (PDMSs), polydimethylsiloxanes comprising alkyl or alkoxy groups, that are pendent and/or at the end of a silicone chain, wherein the alkyl or alkoxy group each comprises from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates.

The fluoro oils that may be used herein include, for example, fluorosilicone oils, fluoropolyethers or fluorosilicones, as described in document EP-A-847 752.

The non-volatile oils may be present in the composition as disclosed herein in an amount ranging, for example, from 0.1% to 20% by weight, such as from 0.1% to 12% by weight, relative to the total weight of the composition.

Additives

The composition disclosed herein may also comprise at least one dyestuff, chosen, for example, from pulverulent dyestuffs, liposoluble dyes and water-soluble dyes. The at least one dyestuff may be present in an amount ranging, for example, from 0.01% to 30% by weight relative to the total weight of the composition.

The pulverulent dyestuffs may be chosen from pigments and nacres.

The pigments may be white or colored, mineral and/or organic, and coated or uncoated. Among the mineral pigments that may be mentioned, examples include titanium dioxide, optionally surface-treated, zirconium oxide, zinc oxide or cerium oxide, and also iron oxide or chromium oxide, manganese violet, ultramarine blue, chromium hydrate and ferric blue. Among the organic pigments that may be mentioned, examples include carbon black, pigments of D&C type and lakes based on cochineal carmine or on barium, strontium, calcium or aluminium.

The nacres may be chosen from white nacreous pigments such as mica coated with titanium or with bismuth oxychloride, colored nacreous pigments such as titanium mica with iron oxides, titanium mica with, for example, ferric blue or with chromium oxide, titanium mica with an organic pigment of the above-mentioned type, and also nacreous pigments based on bismuth oxychloride.

The liposoluble dyes are, for example, Sudan red, D&C Red 17, D&C Green 6, β-carotene, soybean oil, Sudan brown, D&C Yellow 11, D&C Violet 2, D&C Orange 5, quinoline yellow and annatto. The water-soluble dyes are, for example, beetroot juice, methylene blue, the disodium salt of ponceau, the disodium salt of alizarin green, quinoline yellow, the trisodium salt of amaranth, the disodium salt of tartrazine, the monosodium salt of rhodamine, the disodium salt of fuchsin, and xanthophyll.

The composition as disclosed herein may also comprise at least one filler chosen from those that are well known to a person skilled in the art and commonly used in cosmetic compositions. The at least one filler may be mineral or organic, and lamellar or spherical. Mention may be made, for example, of talc, mica, silica, kaolin, powders of polyamide, for instance Nylon® (Orgasol from Atochem), of poly-β-alanine and of polyethylene, powders of tetrafluoroethylene polymers, for instance Teflon®, lauroyllysine, starch, boron nitride, expanded hollow polymer microspheres such as those of polyvinylidene chloride/acrylonitrile, for instance Expancel® (Nobel Industrie), acrylic powders such as Polytrap® (Dow Corning), polymethyl methacrylate particles and silicone resin microbeads (for example Tospearls® from Toshiba), precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids comprising from 8 to 22 carbon atoms such as from 12 to 18 carbon atoms, for example, zinc stearate, magnesium stearate, lithium stearate, zinc laurate or magnesium myristate.

The at least one filler may be present in an amount ranging, for example, from 0.1% to 25% by weight, such as from 1% to 20% by weight, relative to the total weight of the composition.

The composition as disclosed herein may also comprise at least one additive chosen from additives commonly used in cosmetics, such as antioxidants, preserving agents, fragrances, neutralizers, gelling agents, thickeners, vitamins and fibres, and mixtures thereof.

A person skilled in the art will take care to select the optional additional additives and/or the amount thereof such that the advantageous properties of the composition according to the present disclosure are not, or are not substantially, adversely affected by the envisaged addition.

As used herein, the term “fiber” means an object of length L and of diameter D such that L is very much greater than D, wherein D is the diameter of the circle within which the cross section of the fibre is inscribed. For example, the ratio L/D (or shape factor) ranges from 3.5 to 2500, such as from 5 to 500 and further such as from 5 to 150.

The fibers referred to herein have a length ranging, for example, from 1 μm to 10 mm, such as from 0.1 mm to 5 mm and further such as from 0.3 mm to 3 mm.

The fibers that may be used in the composition disclosed herein may be chosen, for example, from rigid or non-rigid fibers, and they may be of synthetic or natural, mineral or organic origin.

Among the fibers that may be used in the composition as disclosed herein, mention may be made, for example, of non-rigid fibers such as polyamide (Nylon®) fibers, or rigid fibers such as polyimide-amide fibers, for instance those sold under the name “Kermel” and “Kermel Tech” by the company Rhodia, or poly(p-phenyleneterephthalamide) (or aramid) fibers sold, for example, under the name Kevlar® by the company DuPont de Nemours.

The compositions as disclosed herein are generally obtained in a standard manner by hot-forming of an emulsion.

For example, the compositions as disclosed herein are obtained by heating the wax, the at least one ionic surfactant and when appropriate, the at least one nonionic surfactant with an HLB of less than or equal to 8 at 25° C. to a temperature higher than the melting point of the wax and not higher than 100° C., until the wax has completely melted, followed by gradual addition of water, nonionic surfactants with an HLB of greater than 8 at 25° C. and, where appropriate, the gelling polymer, brought to a temperature at least equal to the above temperature, with stirring until the mixture has cooled to room temperature. It is also possible to introduce the nonionic surfactant with an HLB of less than or equal to 8 at 25° C. with water.

Water-soluble ingredients may be added to the water used to make the emulsion, or to the emulsion finally obtained.

Similarly, ingredients that optionally present in the composition are added, depending on the case, either to the starting materials or to the finished composition.

In one embodiment, the composition disclosed herein is a mascara.

The composition disclosed herein may be packaged in a cosmetic set comprising a container delimiting at least one compartment which comprises the composition, wherein the container is closed by a closing member.

The container is, for example, combined with an applicator, such as in the form of a brush comprising an arrangement of bristles maintained by a twisted wire. Such a twisted brush is described, for example, in U.S. Pat. No. 4,887,622. It may also be in the form of a comb comprising a plurality of application members, obtained, for example, by moulding. Such combs are described, for example, in French patent FR 2 796 529. The applicator may be integrally attached to the container, as described, for example, in French patent FR 2 761 959. For example, the applicator is integrally attached to a rod which is itself integrally attached to the closing member.

The closing member may be coupled to the container by screwing. Alternatively, the coupling between the closing member and the container is done other than by screwing, such as via a bayonet mechanism, by click-fastening or by tightening. The term “click-fastening” means any system involving the crossing of a bead or cord of material by elastic deformation of a portion, such as the closing member, followed by return to the elastically unconstrained position of the portion after the crossing of the bead or cord.

The container may be at least partially made of thermoplastic material. Examples of thermoplastic materials that may be mentioned include polypropylene and polyethylene.

Alternatively, the container is made of non thermoplastic material, such as glass or metal (or alloy).

The container is, for example, equipped with a drainer arranged in the region of the aperture of the container. Such a drainer makes it possible to wipe the applicator and possibly the rod to which it may be integrally attached. Such a drainer is described, for example, in French patent FR 2 792 618.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The example that follows is given as a non-limiting illustration of the present disclosure.

The amounts indicated are weight percentages and are expressed relative to the total weight of the composition, unless otherwise indicated. The dry extract and the adhesive profile are measured according to the protocols described above.

### EXAMPLE 1

### Mascara

| | |
|---|---|
| 2-Amino-2-methyl-1,3-propanediol | 0.8% |
| Stearic acid (50/50 C₁₆₋₁₈) | 6.6% |
| Carnauba wax | 3.45% |
| Beeswax | 4.3% |
| Paraffin wax | 13.8% |
| Hydroxyethylcellulose quaternized with 2,3-epoxy | 0.1% |
| propyltrimethylammonium chloride | |
| Hydroxyethylcellulose | 0.9% |
| Non-stabilized sodium polymethacrylate at 25% in water | 1% |
| (Darvan 7 from Vanderbilt) | |
| Gum Arabic | 3.4% |
| Polyethylene glycol stearate (40 OE) (Myrj 52P | 0.5% |
| from Uniqema) | |
| 99% triethanolamine | 2.4% |
| Pigments | qs % |
| Preserving agents | qs |
| Sterilized demineralized water | qs 100% |

This composition had a dry extract of 42% and an adhesive profile of less than 3.

The formulation thus obtained had a slippery texture that applied well to the eyelashes. It also had an adhesive power that allowed good eyelash thickening and separating properties.

1. A keratin fiber coating composition comprising, in a cosmetically acceptable medium, at least one ionic surfactant and at least one nonionic surfactant with an HLB of greater than or equal to 8 at 25° C.,
wherein the composition has:
a solids content of greater than or equal to 40% by weight, and
an adhesive profile of less than or equal to 3, wherein said adhesive profile is the ratio of the adhesive power at the time when 20% of said composition has evaporated (AP_{20%}) to the adhesive power at T₀ (AP_{T0}), wherein T₀ means the time of application of the composition.

2. The composition according to claim 1, wherein the adhesive profile ranges from 0.1 to 3.

3. The composition according to claim 2, wherein the adhesive profile ranges from 1.5 to 2.

4. A keratin fiber coating composition comprising, in a cosmetically acceptable medium, at least one ionic surfactant, at least one nonionic surfactant with an HLB of greater than or equal to 8 at 25° C., and at least one paraffin wax, wherein the composition has a solids content of greater than or equal to 40% by weight and is not solid.

5. The composition according to claim 4, wherein the at least one paraffin wax is present in an amount of greater than or equal to 1% by weight, relative to the total weight of the composition.

6. The composition according to claim 5, wherein the at least one paraffin wax is present in an amount of greater than or equal to 5% by weight relative to the total weight of the composition.

7. A keratin fiber coating composition comprising, in a cosmetically acceptable medium, at least one ionic surfactant and at least one nonionic surfactant with an HLB of greater than or equal to 8 at 25° C., wherein the composition has a solids content of greater than or equal to 40% by weight, is free of isomerized jojoba oil, and is not solid.

8. The composition according to claim 1, further comprising at least one wax.

9. The composition according to claim 8, wherein the at least one wax is chosen from waxes of animal origin and waxes of plant origin.

10. The composition according to claim 8, wherein the at least one wax is chosen from hydrocarbon-based waxes; and waxes obtained by catalytic hydrogenation of animal or plant oils comprising at least one fatty chain chosen from linear and branched C₈-C₃₂ fatty chains.

11. The composition according to claim 10, wherein the hydrocarbon-based waxes are chosen from beeswax, lanolin wax and Chinese insect waxes; rice wax, carnauba wax, candelilla wax, ouricurry wax, esparto grass wax, cork fibre wax, sugarcane wax, Japan wax and sumach wax; montan wax; microcrystalline waxes, paraffins and ozokerite; polyethylene waxes; waxes obtained by Fisher-Tropsch synthesis; and waxy copolymers and esters thereof.

12. The composition according to claim 10, wherein the waxes obtained by catalytic hydrogenation of animal or plant oils comprising at least one fatty chain chosen from linear and branched C₈-C₃₂ fatty chains are chosen from hydrogenated jojoba oil, hydrogenated sunflower oil, hydrogenated castor oil, hydrogenated coconut oil and hydrogenated lanolin oil,] bis(1,1,1-trimethylolpropane) tetrastearate, and bis(1,1,1-trimethylolpropane) tetrabehenate.

13. The composition according to claim 8, wherein the at least one wax is present in an amount ranging from 1% to 50% by weight relative to the total weight of the composition.

14. The composition according to claim 13, wherein the at least one wax is present in an amount ranging from 10% to 30% by weight relative to the total weight of the composition.

15. The composition according to claim 1, wherein the at least one nonionic surfactant with an HLB of greater than or equal to 8 is chosen from oxyethylenated fatty alkyl ethers, oxypropylenated fatty alkyl ethers, fatty acid esters of polyethylene glycol, oxyethylenated glycerol ethers, oxypropylenated glycerol ethers, oxyethylenated fatty acid esters of sorbitol ether, oxypropylenated fatty acid esters of sorbitol ether, and copolymers of propylene oxide and of ethylene oxide.

16. The composition according to claim 15, wherein the at lest one nonionic surfactant with an HLB of greater than or equal to 8 is chosen from oxyethylenated ethers of cetearyl alcohol comprising 30 oxyethylene groups, oxyethylene ether of a mixture of C₁₂-C₁₅ fatty alcohols comprising 7 oxyethylene groups, PEG-50 stearate, PEG-40 stearate, PEG-200 glyceryl stearate, polysorbate 60, glyceryl stearate polyethoxylated with 30 ethylene oxide groups, glyceryl oleate polyethoxylated with 30 ethylene oxide groups, glyceryl cocoate polyethoxylated with 30 ethylene oxide groups, glyceryl isostearate polyethoxylated with 30 ethylene oxide groups, glyceryl laurate polyethoxylated with 30 ethylene oxide groups, dimethicone copolyol and dimethicone copolyol benzoate.

17. The composition according to claim 1, wherein the at least one nonionic surfactant with an HLB of greater than or equal to 8 is present in an amount ranging from 0.01% to 40% by weight relative to the total weight of the composition.

18. The composition according to claim 17, wherein the at least one nonionic surfactant with an HLB of greater than or equal to 8 is present in an amount ranging from 0.1% to 25% by weight relative to the total weight of the composition.

19. The composition according to claim 1, wherein the at least one ionic surfactant is chosen from anionic surfactants.

20. The composition according to claim 19, wherein the anionic surfactants are chosen from C₁₆-C₃₀ fatty acid salts; polyoxyethylenated fatty acid salts; phosphoric esters and salts thereof; alkyl ether sulfates; sulfosuccinates; isethionates and acylglutamates, and mixtures thereof.

21. The composition according to claim 1, wherein the at least one ionic surfactant comprises at least one of triethanolamine stearate and 2-amino-2-methyl-1,3-propanediol stearate.

22. The composition according to claim 1, wherein the at least one ionic surfactant is present in an amount ranging from 0.01% to 30% by weight relative to the total weight of the composition.

23. The composition according to claim 22, wherein the at least one ionic surfactant is present in an amount ranging from 0.1% to 15% by weight relative to the total weight of the composition.

24. The composition according to claim 1, wherein the composition comprises from 0.1% to 40% by weight of surfactants relative to the total weight of the composition.

25. The composition according to claim 24, wherein the composition comprises from 0.3% to 20% by weight of surfactants relative to the total weight of the composition.

26. The composition according to claim 1, further comprising at least one film-forming polymer.

27. The composition according to claim 26, wherein the at least one film-forming polymer is chosen from synthetic polymers of free-radical type, synthetic polymers of polycondensate type, and polymers of natural origin.

28. The composition according to claim 26, wherein the at least one film-forming polymer is present in a solids content ranging from 0.1% to 60% by weight relative to the total weight of the composition.

29. The composition according to claim 28, wherein the at least one film-forming polymer is present in a solids content ranging from 1% to 30% by weight relative to the total weight of the composition.

30. The composition according to claim 1, wherein the composition comprises an aqueous phase.

31. The composition according to claim 30, wherein the aqueous phase is present in an amount ranging from 0.1% to 95% by weight relative to the total weight of the composition.

32. The composition according to claim 31, wherein the aqueous phase is present in an amount ranging from 1% to 80% by weight relative to the total weight of the composition.

33. The composition according to claim 1, further comprising at least one dyestuff.

34. The composition according to claim 33, wherein the at least one dyestuff is present in an amount ranging from 0.01% to 30% by weight relative to the total weight of the composition.

35. The composition according to claim 1, wherein the composition has a solids content of greater than or equal to 41% by weight relative to the total weight of the composition.

36. The composition according to claim 35, wherein the composition has a solids content of greater than or equal to 42% by weight relative to the total weight of the composition.

37. The composition according to claim 1, wherein the composition is a mascara.

38. A process for providing a charging makeup on keratin fibers, comprising applying to the keratin fibers a composition comprising, in a cosmetically acceptable medium, at least one ionic surfactant and at least one nonionic surfactant with an HLB of greater than or equal to 8 at 25° C.,
wherein the composition has:
a solids content of greater than or equal to 40% by weight, and
an adhesive profile of less than or equal to 3, wherein said adhesive profile is the ratio of the adhesive power at the time when 20% of said composition has evaporated (AP_{20%}) to the adhesive power at T₀ (AP_{T0}), wherein T₀ means the time of an application of the composition.

39. The method according to claim 38, wherein the keratin fibers are eyelashes.

40. A cosmetic process for treating or making up keratin fibers, comprising applying to the keratin fibers a composition comprising, in a cosmetically acceptable medium, at least one ionic surfactant and at least one nonionic surfactant with an HLB of greater than or equal to 8 at 25° C., wherein the composition has:
a solids content of greater than or equal to 40% by weight, and
an adhesive profile of less than or equal to 3, wherein said adhesive profile is the ratio of the adhesive power at the time when 20% of said composition has evaporated (AP_{20%}) to the adhesive power at T₀ (AP_{T0}), wherein T₀ means the time of an application of the composition.
